(19) United States
(12) Patent Application Publication
(10) Pub. No.: US 2006/0020233 A1
(43) Pub. Date: Jan. 26, 2006

(54) Orthopedic support

(76) Inventor: Arthur M. Bremer, Aiken, SC (US)

Correspondence Address: Stephen R. Greiner, Esquire;GREINER LAW OFFICES, P.C., Suite 110, 6701 Democracy Blvd., Bethesda, MD, 20817, US

(21) Appl. No.: 10/893,961

(22) Filed: Jul. 20, 2004

(51) Int. Cl. A61F 5/00

(52) U.S. Cl. 602/23; 602/62

(57) ABSTRACT

An orthopedic support including a cradle for holding a shoe and guiding the shoe onto the foot of a user. The cradle has an elongated bottom wall for supporting the shoe. A retaining wall extends upwardly from the periphery of the bottom wall for holding the shoe on the bottom wall. The retaining wall has, adjacent the rear end of the bottom wall, an upward extension that serves as a guide for moving a leg toward the shoe. A handle is secured to the cradle and extends upwardly from it. A prop is secured to the cradle for increasing the elevation of the front end of the bottom wall relative to its rear end.

### FIELD OF THE INVENTION

The present invention relates generally to apparel apparatus and, more particularly, such apparatus for putting on and removing boots and shoes.

### BACKGROUND OF THE INVENTION

It is difficult for many people to dress themselves. Conditions such as arthritis, obesity and stroke can make it hard for some individuals to twist, turn or bend their bodies and limbs to enter articles of apparel. Putting on shoes is particularly tough and, for some, the task is impossible to accomplish without assistance.

To don shoes, an individual must typically bend to fetch the shoes from a storage place on the floor. Then, he must grasp the shoes in his hands as he slips his feet into them. Finally, if the shoes are provided with laces, they must be tied. Many infirm individuals simply lack the flexibility and dexterity to retrieve and put on shoes. In fact, bending over may, for some, pose a risk of serious bodily harm from falling or fainting.

### SUMMARY OF THE INVENTION

In light of the problems associated with donning and doffing shoes that are encountered by some individuals with limited flexibility and dexterity, it is a principal object of the invention to provide an orthopedic support that permits the easy manipulation of a shoe (or leg brace). The support firmly holds the shoe as it is being moved into a comfortable position for wear and guides the leg of a user toward the shoe. The assistance of other people to put on, and take off, shoes is no longer required with the orthopedic support.

It is another object of the invention to provide an orthopedic support of the type described that can be manually adjusted to accommodate shoes of different size. The adjustment can be accomplished without tools of any sort and with minimal instruction. In fact, the support is particularly intuitive to use.

It is a further object of the invention to provide an orthopedic support of the type described that features a prop that tilts the support rearwardly to ease the entry of a foot of a seated user into a shoe. The prop also serves to stabilize the orthopedic support and prevent it from being easily tipped over.

It is an object of the invention to provide improved elements and arrangements thereof in an orthopedic support for the purposes described that is lightweight in construction, inexpensive to manufacture, and dependable in use.

Briefly, the orthopedic support in accordance with this invention achieves the intended objects by featuring a cradle for holding a shoe and guiding the shoe onto the foot of a user. The cradle has an elongated bottom wall for supporting the shoe. A retaining wall extends upwardly from the periphery of the bottom wall for holding the shoe on the bottom wall. The retaining wall has, adjacent the rear end of the bottom wall, an upward extension for guiding a user's leg toward the shoe. The cradle is also divided into two parts: a heel portion and a toe portion that are movably attached to one another. A handle is secured to the cradle and extends upwardly from it. A prop is secured to the cradle for increasing the elevation of the front end of the bottom wall relative to its rear end.

The foregoing and other objects, features and advantages of the present invention will become readily apparent upon further review of the following detailed description of the preferred embodiment as illustrated in the accompanying drawings.

### BRIEF DESCRIPTION OF THE DRAWINGS

The present invention may be more readily described with reference to the accompanying drawings, in which:

FIG. 1 is a side elevational view of an orthopedic support in accordance with the present invention with the front portion broken away to reveal details thereof.

FIG. 2 is a perspective view of the orthopedic support of FIG. 1.

FIG. 3 is a bottom view of the front portion of the orthopedic support.

Similar reference characters denote corresponding features consistently throughout the accompanying drawings.

### DETAILED DESCRIPTION OF THE PREFERRED EMBODIMENT

Referring now to the FIGS., an orthopedic support in accordance with the present invention is shown at 10. Support 10 includes a cradle 12 for holding a shoe 14 and guiding shoe 14 onto a user's leg 16. A handle 18 extends upwardly from cradle 12 so that a user can easily move cradle 12 for access to shoe 14. A prop 20 extends downwardly from cradle 12 to tilt cradle 12 rearwardly and stabilize cradle 12 during use.

Cradle 12 includes an elongated bottom wall 22 with an outline resembling that of shoe 14. A retaining wall 24 extends upwardly from the periphery of bottom wall 22 to a height that is generally about one-half that of shoe 14. The rear end of wall 22, however, is provided with an upward extension 26 that serves as a guide for moving leg 16 toward shoe 14. Extension 26 has a C-shaped cross section with a concave front surface 28 that opens forwardly to retain leg 16 in alignment with bottom wall 22 as leg 16 is slid downwardly along extension 26 toward shoe 14.

Cradle 12 is divided into a heel portion 30 and a toe portion 32 that can be moved relative to one another so that the length of cradle 12 can be varied to accommodate shoes that are larger and smaller than shoe 14. Heel portion 30 includes the rear part 22a of bottom wall 22 and the rear part 24a of retaining wall 24 along with upward extension 26. Toe portion 32 has the front part 22b of bottom wall 22 and the front part 24b of retaining wall 24.

An adjustable mounting bracket 34 secures toe portion 32 to heel portion 30. Mounting bracket 34 includes a rail 36 attached at its rear end by rivets 38 or like fasteners to heel portion 30 and, more specifically, to the underside of rear part 22a of bottom wall 22. Rail 36 extends forwardly from heel portion 30 and terminates in a pair of parallel branches 40 separated by a linear slot 42. Mounting bracket 34 also includes a pair of threaded fasteners 44 secured to toe portion 32. As shown, the heads 46 of fasteners 44 are secured to front part 22b of bottom wall 22 with the threaded shafts 48 of fasteners 44 extending downwardly beneath front part 22b. Wing nuts 50 are rotatably secured to threaded shafts 48.

Threaded shafts 48 of fasteners 44 are slidably positioned within slot 42 and retain wing nuts 50 against the undersides of branches 40. When wing nuts 50 are loosened, toe portion 32 can be pulled away from heel portion 30, for example, as is illustrated in moving toe portion 32 from the solid line position in FIG. 3 to the broken line position. Retightening wing nuts 50 selectively locks toe portion 32 in an extended position on rail 36.

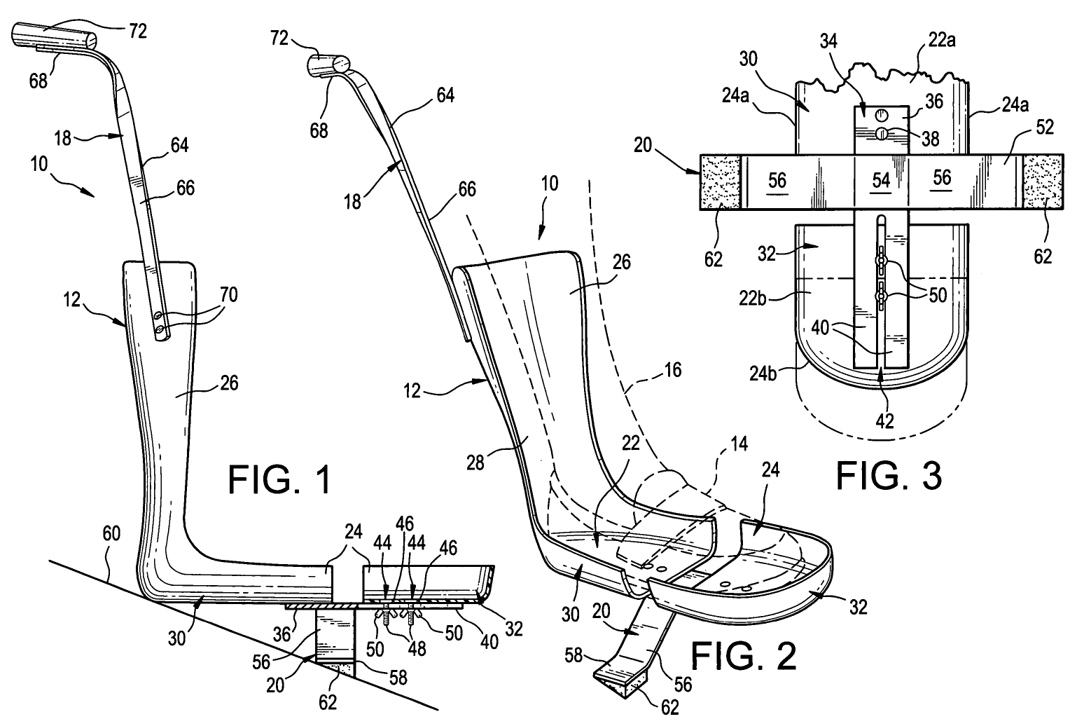

Prop 20 includes an elongated bar 52 that has been deformed to elevate toe portion 32. In this regard, bar 52 has a center portion 54 that is affixed to the underside of rail 36 beneath heel portion 30. A pair of leg portions 56 extends outwardly and downwardly from center portion 54. One of a pair of foot portions 58, oriented substantially parallel to the bottom of portion 54 and cradle 12, extends outwardly from each of leg portions 56. When positioned on a horizontal supporting surface 60 (inclined for the sake of convenience in FIG. 1), the rear ends of foot portions 58 and of rear part 22a provide a stable, three-point suspension for cradle 12.

Secured to the bottoms of foot portions 58 are triangular foam pads 62. Pads 62 maintain the stability of support 10 by evenly distributing loads from bar 52 to supporting surface 60. Furthermore, pads 62 prevent bar 52 from scuffing floors.

Handle 18 has an elongated shaft 64 with an upright piece 66 connected to a reclined piece 68 in a configuration resembling an inverted “L”. The bottom of upright piece 66 is secured by rivets 70 or other suitable means to the top of extension 26. Reclined piece 68 extends rearwardly from upright piece 66 with which it is integrally formed. A cylindrical handgrip 72 is affixed to the top of reclined piece 68 for ease in grasping handle 18.

Use of orthopedic support 10 is straightforward. First, a user places shoe 14 atop bottom wall 22 of cradle 12. (If bottom wall 22 is of insufficient length to receive shoe 14, wing nuts 50 are temporarily loosened to permit toe portion 32 to be pulled away from heel portion 30.) Then, while holding onto handle 18, support 10 is lowered to surface 60 upon which the user should be standing. Next, the user slides the back of leg 16 downwardly along concave surface 28 of extension 26 and inserts his foot into shoe 14. The user can now withdraw his shorn foot from support 10 and walk away in a normal manner. The entire process requires only a few seconds to complete.

A user doffs shoe 14 by reversing the steps noted above. Once shoe 14 is removed it can be positioned in a convenient location for future access when needed. Support 10 can be similarly located and may be kept on horizontal surface 60 if desired. Handle 18 has a sufficient length that a user need not bend or stoop to pick it up. Thus, orthopedic support 10 is always ready for immediate reuse.

While the invention has been described with a high degree of particularity, it will be appreciated by those skilled in the art that modifications may be made thereto. Therefore, it is to be understood that the present invention is not limited to the sole embodiment described above, but encompasses any and all embodiments within the scope of the following claims.

I claim:

1. An orthopedic support, comprising:
a cradle for holding a shoe and guiding the shoe onto the foot of a user, the cradle includes:
an elongated bottom wall for supporting the shoe, said bottom wall having a front end and a rear end opposite said front end;
a retaining wall extending upwardly from the periphery of said bottom wall for holding the shoe on said bottom wall, said retaining wall having, adjacent said rear end of said bottom wall, an upward extension projecting upwardly from said retaining wall and serving as a guide for moving a leg toward the shoe;
a handle being secured to said cradle and extending upwardly therefrom; and,
a prop being secured to said cradle for increasing the elevation of said front end of said bottom wall relative to said rear end.

2. The orthopedic support according to claim 1 wherein said upward extension has a C-shaped cross section with a concave front surface that opens forwardly to retain the leg of a user in alignment with said bottom wall as the foot of a user is slid downwardly along said upward extension toward the shoe.

3. The orthopedic support according to claim 1 wherein said cradle includes a heel portion and a toe portion secured together by an adjustable mounting bracket whereby the length of said cradle can be selectively varied.

4. The orthopedic support according to claim 3 wherein said adjustable mounting bracket includes:
a rail being secured to said heel portion and extending forwardly therefrom; said rail terminates in a pair of parallel branches separated by a slot remote from said heel portion;
a plurality of threaded fasteners being secured to said toe portion, of said threaded fasteners having a threaded shaft slidably positioned within said slot in said rail; and,
a plurality of wing nuts each being rotatably secured to a respective one of said threaded shafts for clamping said parallel branches of said rail.

5. The orthopedic support according to claim 1 wherein said handle includes:
an elongated shaft with an upright piece being connected to a reclined piece in an inverted L-shaped configuration, the bottom of said upright piece being secured to the top of said upward extension of said retaining wall, and said reclined piece extending rearwardly from said upright piece; and,
a handgrip being affixed to said reclined piece.

6. The orthopedic support according to claim 1 wherein said prop includes:
an elongated bar having:
a center portion secured to the bottom of said bottom wall, said center portion having opposite sides;
a pair of leg portions, each extending outwardly and downwardly from a respective one of said opposite sides of said center portion; and,
a pair of foot portions, each extending outwardly from a respective one of said leg portions; and,
a pair of resilient pads, each being secured to a respective one of said foot portions for preventing said bar from scuffing floors.

7. An orthopedic support, comprising:
a cradle for holding a shoe and guiding the shoe onto the shoe of a user, the cradle includes:
an elongated bottom wall for supporting the shoe, said bottom wall having a front end and a rear end opposite said front end;
a retaining wall extending upwardly from the periphery of said bottom wall for holding the shoe on said bottom wall, said retaining wall having, adjacent said rear end of said bottom wall, an upward extension projecting upwardly from said retaining wall and serving as a guide for moving a leg toward the shoe;
said cradle being divided laterally into a heel portion and a toe portion movably attached to one another, said heel portion having said rear end of said bottom wall and said toe portion having said front end of said bottom wall and said upward extension;
a handle being secured to said cradle and extending upwardly therefrom; and,
a prop being secured to said cradle for increasing the elevation of said front end of said bottom wall relative to said rear end.
